(19) United States
(12) Patent Application Publication
(10) Pub. No.: US 2017/0189330 A1
(43) Pub. Date: Jul. 6, 2017

(54) COUGH SYRUP CONTAINING AMBROXOL HYDROCHLORIDE

(71) Applicant: BOEHRINGER INGELHEIM INTERNATIONAL GMBH, Ingelheim am Rhein (DE)

(72) Inventors: Bernd PLOHMANN, Oberhoefen (DE); Katrin BUSZELLO, Birkenhard (DE); Uwe SCHEURING, Ochsenhausen-Hattenburg (DE); Annette ZAMPONI, Biberach an der Riss (DE)

(21) Appl. No.: 15/313,159

(22) PCT Filed: May 19, 2015
(86) PCT No.: PCT/EP2015/060991

§ 371 Date: Nov. 22, 2016

(30) Foreign Application Priority Data
May 23, 2014 (EP) 14169642.7

(51) Int. Cl. A61K 9/00; A61K 31/136

(52) U.S. Cl. CPC A61K 9/0056; A61K 31/136

(57) ABSTRACT

The present invention relates to cough syrups containing ambroxol hydrochloride, characterised in that they are substantially free from glycerol, and their use for mucolytic treatment in acute and chronic diseases of the bronchi and/or lungs.

The present invention relates to cough syrups containing ambroxol hydrochloride, characterised in that they are substantially free from glycerol, and their use for mucolytic treatment in acute and chronic diseases of the bronchi and/or lungs.

Ambroxol is a metabolite of bromhexine (Bisolvon®), which is itself derived from vasicine, a plant extract from the Malabar nut plant.

Ambroxol is an active substance with a local analgesic, mucolytic and anti-inflammatory effect which is used to treat sore throats and respiratory diseases accompanied by viscous mucus formation, e.g. in acute aggravations of chronic bronchitis, asthmatic bronchitis and bronchial asthma.

Ambroxol is usually present in medicaments in the form of ambroxol hydrochloride. Ambroxol is commercially available, inter alia, in the form of suckable lozenges, capsules, as an inhalable solution, syrup and linctus.

When stored in the standard commercial compositions ambroxol hydrochloride is subject to a slow process of decomposition.

The problem of the present invention was to provide a cough syrup containing ambroxol hydrochloride which is characterised by a particularly high storage stability, i.e. by a particularly low rate of decomposition of ambroxol hydrochloride.

According to the invention this problem is solved by a cough syrup containing ambroxol hydrochloride which differs from the standard commercial cough syrups in that it is substantially free from glycerol. In another embodiment of the invention, this problem is solved by a cough syrup containing ambroxol hydrochloride which differs from the standard commercial cough syrups in that it is substantially free from glycerol and sugar alcohols.

The cough syrups according to the invention are surprisingly characterised by a very low decomposition rate of ambroxol hydrochloride. Consequently there is virtually no need to add any stabilisers.

Of course, the cough syrups according to the invention are suitable for conventional use without any restrictions. Consequently, a further object of the invention relates to the use of a cough syrup according to the invention for mucolytic treatment in acute and chronic diseases of the bronchi and/or lungs.

Within the scope of the present invention the term “ambroxol hydrochloride” denotes trans-4-((2,4-dibromoanilin-6-yl)-methylamino)-cyclohexanol hydrochloride.

Usually, the cough syrup according to the invention contains ambroxol hydrochloride in an amount of 0.1 to 1.0 g, based on 100 ml of the cough syrup. The specified amount of ambroxol hydrochloride relates in each case to the amount of the salt used. Preferably, the cough syrups according to the invention contain ambroxol hydrochloride in an amount of 0.1 to 1.0 g, for example in an amount of 0.3 g or 0.6 g, based in each case on 100 ml of the cough syrup.

One particular embodiment of the present invention relates to a cough syrup according to the invention which contains ambroxol hydrochloride as the sole active substance, i.e. a so-called monopreparation.

The cough syrup according to the invention is characterised in that it is substantially free from glycerol.

The term “substantially free from glycerol” is to be understood as meaning that glycerol is used in an amount which has no appreciable effect on the properties of the cough syrup. Usually, the cough syrup according to the invention will contain glycerol in an amount of less than 5 g based on 100 ml of the cough syrup. Preferably, the amount of glycerol in the cough syrup according to the invention is less than 1 g and particularly preferably less than 0.5 g, based in each case on 100 ml of the cough syrup. One particular embodiment of the invention relates to a cough syrup which is free from glycerol.

A preferred embodiment of the present invention relates to a cough syrup according to the invention, characterised in that it is substantially free from sugar alcohols.

Within the scope of the present invention, the term “sugar alcohol” denotes compounds of the general formula HOCH₂[CH(OH)]ₙCH₂OH (wherein n≧1), which may be obtained by reduction of a saccharide, particularly a mono- or disaccharide. Conventional sugar alcohols as additives in pharmaceutical compositions are, for example, sorbitol, xylitol, maltitol, isomalt, mannitol, threitol, erythritol and arabitol.

The term “substantially free from sugar alcohols” is to be understood as meaning that sugar alcohols are used in an amount which has no appreciable effect on the properties of the cough syrup. Usually, the cough syrup according to the invention will contain sugar alcohols in an amount of less than 10 g based on 100 ml of the cough syrup. Preferably, the amount of sugar alcohols in the cough syrup according to the invention is less than 5 g and particularly preferably less than 1 g, based in each case on 100 ml of the cough syrup. One particular embodiment of the invention relates to a cough syrup which is free from sugar alcohols.

Usually, the cough syrup according to the invention contains a suitable thickener. The cough syrup according to the invention can be adjusted to the required viscosity using this. Consequently, the cough syrup according to the invention contains the thickener in an amount that is suitable for creating the desired viscosity. Usually, the cough syrup according to the invention contains the thickener in an amount of 0.001 to 10 g, preferably 0.005 to 5 g, particularly preferably 0.01 to 1 g, based in each case on 100 ml of the cough syrups according to the invention.

Suitable thickeners are selected for example from among hydroxypropylmethylcellulose (HPMC), hydroxyethylcellulose (HEC), hydroxypropylcellulose (HPC), methylcellulose (MC), carboxymethylcellulose (CMC), and methylethylcellulose (MEC). Preferably, the thickeners are selected from among hydroxypropylmethylcellulose (HPMC), hydroxyethylcellulose (HEC) and hydroxypropylcellulose (HPC). In particular, the thickener used according to the invention is hydroxyethylcellulose.

The cough syrup according to the invention is usually characterised by a viscosity in the range from 1 mPa·s to 30 Pa·s at a temperature of 20° C. Preferably, the viscosity is in the range from 10 mPa·s to 5 Pa·s and particularly preferably in the range from 30 mPa·s to 1 Pa·s. The values stated are based on determination of the viscosity at a temperature of 20° C. with a ball-drop viscosimeter using the method of the European Pharmacopoeia (6th edition, page 84, chapter 2.2.49).

Usually, the cough syrup according to the invention contains a suitable sweetener. Preferably, according to the invention, sweeteners which are different from sugar alcohols are used, i.e. in this embodiment the cough syrup according to the invention is free from sugar alcohols.

Suitable sweeteners are selected, for example, from among sucralose, acesulfame, aspartame, cyclamate, saccharine, isomalt, maltitol, xylitol, lactitol, erythritol, alitame, thaumatin and neohesperidin dihydrochalcone. Sucralose, acesulfame, aspartame, cyclamate, saccharine, alitame, thaumatin and neohesperidin dihydrochalcone are preferred. The sweetener sucralose is particularly preferred.

The cough syrup according to the invention usually contains the sweetener in an amount of 0.01 g to 10 g, based on 100 ml of the cough syrup. Preferably, the cough syrup according to the invention contains the sweetener in an amount of 0.05 to 1 g and particularly preferably in an amount of 0.05 to 0.5 g, based in each case on 100 ml of the cough syrup according to the invention.

Usually, the cough syrup according to the invention contains a suitable preservative.

The term “preservative” as used herein does not encompass any sugar alcohols, which are also known to have a preservative effect.

Suitable preservatives include, for example, benzoic acid, sorbic acid, sulphurous acid or the salts thereof. Benzoic acid, in particular, has proved a suitable preservative for cough syrups containing ambroxol.

The cough syrup according to the invention usually contains the preservative in an amount of 0.005 to 1.0 g based on 100 ml of the cough syrup. Preferably, the cough syrup according to the invention contains the preservative in an amount of 0.01 to 0.5 g and particularly preferably in an amount of 0.02 to 0.1 g, based in each case on 100 ml of the cough syrup.

The cough syrups according to the invention are usually aqueous compositions. The term “aqueous compositions” as used herein preferably refers to compositions wherein the solvents consist of at least 80% by weight and particularly preferably at least 90% by weight of water. One particular embodiment relates to cough syrups in which the solvent consists exclusively of water. Such cough syrups are alcohol-free and thus fundamentally suitable for use in children.

The proportion of solvent, particularly water, in the cough syrups according to the invention is usually at least 50% by weight. The cough syrups according to the invention are usually characterised by a water content of at least 77% by weight, preferably at least 90 and particularly preferably at least 95% by weight.

One particular embodiment of the present invention relates to a cough syrup consisting of:

a) ambroxol hydrochloride in an amount of 0.1 g to 1.0 g,

b) thickener in an amount of 0.01 g to 1.0 g,

c) sweetener in an amount of 0.01 g to 10 g,

d) preservative in an amount of 0 to 1.0 g and

d) flavourings in an amount of 0 to 10 g and

e) water ad 100 ml.

With regard to the preferred amounts and nature of the components of this particular embodiment the remarks made earlier apply.

Suitable flavourings are known to the skilled man.

The cough syrups according to the invention are produced by conventional formulation methods. It is not critical whether the components of the cough syrups according to the invention are mixed together at the same time or successively. The order in which this is done is also of no consequence. The components of the cough syrups according to the invention may be provided in pure form, in the form of solutions, or in the form of partial compositions which already contain a number of ingredients of the cough syrups according to the invention.

Due to their high stability, the cough syrups according to the invention are suitable for packing in all the conventional packaging forms.

The invention will hereinafter be described in more detail by means of non-limiting examples.

### EXAMPLES

In order to investigate their stability, two cough syrups according to the invention (composition (A) and (B)) and two compositions corresponding to standard commercial cough syrups (compositions (C) and (D)) were transferred into standard commercial brown glass bottles. The stability of the composition under controlled storage conditions (6 months at 40° C., 75% rel. humidity) was evaluated on the basis of the amount of three decomposition products of ambroxol hydrochloride present. For this purpose, the content of decomposition products (1) to (3) in the samples was determined by HPLC and UV detection.

| | (A) | (B) | (C) | (D) |
|---|---|---|---|---|
| Composition | content [mg/ml] | | | |
| Ambroxol hydrochloride | 3 | 6 | 3 | 3 |
| Benzoic acid | 0.5 | 0.5 | 1.7 | 1.7 |
| Hydroxyethylcellulose | 4 | 4 | 2 | 2 |
| Acesulfame potassium | — | — | 1 | 1 |
| Sucralose | 1 | 1.26 | — | — |
| Sorbitol | — | — | 350 | 350 |
| (70% solution) | | | | |
| Glycerol | — | — | 150 | 150 |
| (85% solution) | | | (synthetic) | (vegetable) |
| Flavourings | 4.4 | 4.8 | 2.2 | 2.4 |
| Water | ad 1 ml | ad 1 ml | ad 1 ml | ad 1 ml |

Decomposition product (1): Molecular weight: 424.6 g/mol

C₁₄H₁₆Br₂N₂O*HCl Total formula (empirical)

Decomposition product (2): Molecular weight: 426.6 g/mol;

C₁₄H₁₈Br₂N₂O*HCl Total formula (empirical)

Decomposition product (3): Molecular weight: 487.6 g/mol

C₁₆H₂₂Br₂N₂O₃*HCl Total formula (empirical)

The results of this investigation are summarised below.

Composition (A)

| | Amount (based on 3 mg |
|---|---|
| Decomposition product | ambroxol hydrochloride/ml) |
| A | <0.05% |
| B | 0.07% |
| C | <0.05% |
| Total | 0.07% |

Composition (B)

| | Amount (based on 6 mg |
|---|---|
| Decomposition product | ambroxol hydrochloride/ml) |
| A | <0.05% |
| B | <0.05% |
| C | <0.05% |
| Total | <0.05% |

Composition (C) (Comparison example)

| | Amount (based on 3 mg |
|---|---|
| Decomposition product | ambroxol hydrochloride/ml) |
| A | 0.19% |
| B | 0.17% |
| C | 0.16% |
| Total | 0.52% |

Composition (D) (Comparison example)

| | Amount (based on 3 mg |
|---|---|
| Decomposition product | ambroxol hydrochloride/ml) |
| A | 0.27% |
| B | 0.32% |
| C | 0.12% |
| Total | 0.71% |

The test results show that the cough syrups according to the invention are distinguished from the standard commercial compositions containing ambroxol by a particularly low rate of decomposition of ambroxol hydrochloride.

1. A cough syrup containing ambroxol hydrochloride, characterised in that it is substantially free from glycerol.

2. The cough syrup according to claim 1, characterised in that it is also substantially free from sugar alcohols.

3. The cough syrup according to claim 1, also containing a suitable thickener.

4. The cough syrup according to claim 1, having a viscosity in the range from 1 mPa·s to 30 Pa·s at a temperature of 20° C.

5. The cough syrup according to claim 1, containing a suitable sweetener.

6. The cough syrup according to claim 5, present in an amount of 0.01 g to 10 g based on 100 ml of the cough syrup.

7. The cough syrup according to claim 1, having a water content of at least 77% by weight.

8. The cough syrup according to claim 1, consisting of:
a) ambroxol hydrochloride in an amount of 0.1 g to 1.0 g,
b) thickener in an amount of 0.01 g to 1.0 g,
c) sweetener in an amount of 0.01 g to 10 g,
d) preservative in an amount of 0 to 1.0 g and
d) flavourings in an amount of 0 to 10 g and
e) water ad 100 ml.

9. (canceled)
